(19) United States
(12) Patent Application Publication
(10) Pub. No.: US 2016/0230192 A1
(43) Pub. Date: Aug. 11, 2016

(54) TRITERPENOIDS HIGH YIELDING STRAIN OF ANTRODIA CINNAMOMEA AND USE THEREOF

(71) Applicant: SHENZHEN RENTAI BIOLOGICAL TECHNOLOGY CO., LTD., Shenzhen, Guangdong (CN)

(72) Inventors: Zongjie Zhao, Shenzhen (CN); Haitao Xie, Shenzhen (CN); Qingsong Cai, Shenzhen (CN)

(21) Appl. No.: 14/413,420

(22) PCT Filed: Aug. 20, 2013
(86) PCT No.: PCT/CN2013/081863

§ 371 Date: Jan. 8, 2015

(51) Int. Cl. C12P 5/00; C12R 1/645

(52) U.S. Cl. CPC C12P 5/007; C12R 1/645

(57) ABSTRACT

The present invention discloses a triterpenoids high yielding strain of Antrodia cinnamomea and use thereof. The accession number of the Antrodia cinnamomea strain with a high triterpenoids yield is CCTCC M2013359. As for the strain mycelium, the growth rate is fast, the triterpenoids output is high, and the genetic properties are stable, so the strain mycelium is suitable for industrialized fermentation. Compared with the original strain, the mycelium output under a liquid culturing condition is increased by more than 1.5 times, and the intracellular triterpenoids output is increased by 2 to 3 times, the fermentation period is shortened to 65 hours compared with the original more than 10 days; and the total triterpenoids output under a solid culturing condition is increased by 1 to 2 times, and the fermentation period is shortened to 10 days compared with the original more than 90 days.

### BACKGROUND

1. Technical Field

The present invention relates to the field of biological engineering, and in particular, to a novel triterpenoids high yielding strain of Antrodia cinnamomea bred through ultraviolet-induced mutation of a protoplast, and the use thereof.

2. Related Art

Antrodia cinnamomea is a rare medicinal fungus originating in Taiwan, and is honored as “miracle anticancer drug”, “master of liver protectors” and “master of antidotes” due to extremely high medicinal value, and is the most expensive medicinal fungus at present. Modern medical researches confirm that the main active ingredients of Antrodia cinnamomea are triterpenoids. However, wild Antrodia cinnamomea is rare, artificially cultured Antrodia cinnamomea fruiting body has triterpenoids ingredients similar to the wild Antrodia cinnamomea, but the fruiting body grows slowly and the market demands cannot be met. Presently, artificial fermentation by adopting a modern biotechnology is the optimal way to obtain triterpenoids from Antrodia cinnamomea. Artificial fermentation is mainly classified into solid fermentation and submerged fermentation. The period of solid fermentation of Antrodia cinnamomea is long (90 to 180 days), and the total triterpenoids content in the solid fermentation product is about 5%, which is far less than the total triterpenoids content of 10% to 45% in the wild fruiting body; the period of liquid fermentation of Antrodia cinnamomea is relatively shorter (7 to 14 days), but the product substantially does not contain the triterpenoids. Therefore, the two artificial fermentation methods have the disadvantages of either long fermentation period or low triterpenoids output. Strain is anintrinsic factor that deter mines the fermentation level, and for many medicinal fungi, outputs of pharmaceutically active ingredients are significantly increased through mutation breeding, and industrialized production of medicinal fungi is achieved. Li Gang (2001) and Li Yingying et al (2011) respectively obtained excellent ganoderma strains with high polysaccharide and triterpenoids yields through conventional mutation breeding; Zhang Rijun (2004) obtained a bacillus with a high protease yield through combined chemical and physical mutation; and Ying Hanjie et al (2007) obtained a penicillium citrinum with a high nuclease yield by injecting a low-energy particle beam into the spore. There are many similar studies, and it can be seen that, mutation breeding is an efficient means for improving the active matter output of edible and medicinal fungi.

Until now, there are few reports on studies of mutation breeding of Antrodia cinnamomea, with the only literature document shows that Tian Xuemei et al (2012) from Shandong Provincial Key Laboratory of Applied Fungi, Qingdao Agricultural University, obtained 5 Antrodia cinnamomea mutant strains through physical mutation, in which the strain having a code of 327 is a strain with high polysaccharide and protein yields, and the polysaccharide output and the protein output are increased by 238.2% and 10.33% respectively, compared with the original strain. Gg is a strain with a high triterpenoids yield with the triterpenoids output is increased by 57.86% compared with the original strain and the total triterpenoids output under the liquid fermentation condition is 18 mg/L, equivalent to the total triterpenoids content of dry mycelium of 0.47%. However, in fact, the triterpenoids content is not high, and the situation remains far away from the industrialization requirements.

Therefore, the prior art still needs to be improved and developed.

### SUMMARY

### Technical Problem

In view of the disadvantages of the prior art, objectives of the present invention are to provide a triterpenoids high yielding strain of Antrodia cinnamomea and the use thereof, so as to solve the problem of low triterpenoids output in existing artificial fermentation of Antrodia cinnamomea strains.

### Solutions for the Problem

### Technical Solutions

In the solutions of the present invention, a method for ultraviolet-induced mutation of a protoplast is adopted. Strain mutation breeding includes physical mutation and chemical mutation. Common physical mutation methods include ultraviolet-induced mutation, microwave-induced mutation, spatial mutation, ⁶⁰Coγ-ray irradiation mutation, X-ray-induced mutation and ion injection mutation. Chemical mutagens mainly include alkylation agents, base analogues, hydroxyla mine and acridine dyes. The ultraviolet-induced mutation has the advantages of simple operation, high mutation efficiency, high reproducibility, and easily available mutation source, and has become a conventional mean for mutation breeding of various biomaterials.

Antrodia cinnamomea belongs to macro fungi and has a tough and tensile spore shell. If the spore is directly used as a mutagenic material, high-intensity mutation treatment is required, and moreover, the success rate is low, because even if mutation is successful, the spore is difficult to ger minate under artificial conditions. Meanwhile, most of Antrodia cinnamomea mycelia are chain composite cell body, and thus mutated single cell cannot be screened out. Therefore, the mycelium is also not an appropriate mutagenic material. The advanced method for producing mutants of Antrodia cinnamomea is indirect mutation of a protoplast, in which the cell wall is removed using a biological enzymatic method to prepare a protoplast. The protoplast is sensitive to ambient environment and is easily mutated after mutagenic treatments. Moreover, by using protoplast, regenerated colonies are developed from single cells, and can be easily screened. The regenerated strains can maintain main biological properties of the original strain, significantly shorten the breeding period, have stable biological characteristics, and are resistant to degeneration.

As for the selection of original strain, the original strain used in mutation breeding is very critical. Presently, there are more than a dozen of Antrodia cinnamomea strains in culture collection agencies worldwide. Most of the strains are isolated from fruiting bodies of wild Antrodia cinnamomea in the nature or tree holes of cinnamomum kanehirai, and mycelia of these strains grow weakly and slowly in a flat-plate and liquid media. In the present invention, an Antrodia cinnamomea strain ATCC 200183 is used as the original strain. The mycelium of strain ATCC 200183, which is relatively thick and strong, and grows relatively quicker in flat-plate and liquid media, is an ideal original strain for screening an ideal strain of Antrodia cinnamomea.

The purpose of the present invention is to obtain an Antrodia cinnamomea strain with a high triterpenoids yield under liquid culturing condition. During screening, the time for the Antrodia cinnamomea mycelium growing to fulfill a flat-plate and the total triterpenoids output of shake-culturing mycelium fermentation are taken as indexes, Antrodia cinnamomea protoplast is repeatedly mutated by using ultraviolet rays to obtain a novel Antrodia cinnamomea strain with a high triterpenoids yield.

In view of the above, the present invention provides a method for breeding a novel Antrodia cinnamomea strain with a high triterpenoids yield through ultraviolet-induced mutation of a protoplast, and a novel Antrodia cinnamomea strain with a high triterpenoids yield produced by using this method. The novel Antrodia cinnamomea strain was deposited under the accession number of CCTCC M2013359. The mutation steps include: using an Antrodia cinnamomea strain ATCC 200183 as an original strain, shake-culturing mycelium to prepare a protoplast, carrying out ultraviolet-induced mutation; coating on an MEA flat-plate, selecting a single colony for shaking culture, taking the mycelium output and the intracellular total triterpenoids content as indexes, and finally obtaining a novel Antrodia cinnamomea strain with a high triterpenoids yield. The mycelium of the obtained novel strain has a fast growth rate, a high triterpenoids output, and stable genetic properties, and is suitable for industrial fermentation. Compared with the original strain ATCC 200183, the novel strain provides the following advantages:

the mycelium output under liquid culturing condition is increased by more than 1.5 times;

the intracellular triterpenoids output is increased by 2 to 3 times;

the fermentation period is shortened to 65 hours compared with the original of more than 10 days;

the total triterpenoids output under solid culturing condition is increased by 1 to 2 times; and

the fermentation period is shortened to 10 days compared with the original of more than 90 days.

The present invention further provides a use of the above novel Antrodia cinnamomea strain with a high triterpenoids yield for producing triterpenoids through fermentation.

### Beneficial Effects of the Present Invention

### Beneficial Effects

Beneficial effects: The present invention discloses a method for breeding a novel Antrodia cinnamomea strain with a high triterpenoids yield through ultraviolet-induced mutation of a protoplast and an Antrodia cinnamomea strain with a high triterpenoids yield breed by using this method, where the accession number of the strain is CCTCC M2013359. The mutation steps include: using an ATCC 200183 Antrodia cinnamomea strain as an original strain, shake-culturing mycelium to prepare a protoplast, carrying out ultraviolet-induced mutation; coating on an MEA flat-plate, selecting a single colony for shaking culture, taking the mycelium output and the intracellular total triterpenoids content as indexes, and finally obtaining the Antrodia cinnamomea strain with a high triterpenoids yield. As for the strain mycelium, the growth rate is fast, the triterpenoids output is high, and the genetic properties are stable, so the strain mycelium is suitable for industrialized fermentation.

### BRIEF DESCRIPTION OF THE DRAWINGS

FIG. 1 shows the mycelium dry weight and the triterpenoids output per liter of fermentation broth of each mycelium group after fermentation was completed and the fermentation broth was dried at 40° C. according to Example 2 of the present invention;

FIG. 2 is a data comparison chart of determination results of the mycelium dry weight according to Example 2 of the present invention;

FIG. 3 is a data comparison chart of determination results of the triterpenoids output according to Example 2 of the present invention;

FIG. 4 shows the mycelium dry weight and the triterpenoids output per liter of fermentation broth of each mycelium group after fermentation was completed and the fermentation broth was dried at 40° C. according to Example 3 of the present invention;

FIG. 5 is a data comparison chart of determination results of the mycelium dry weight and the triterpenoids content according to Example 3 of the present invention;

FIG. 6 shows the triterpenoids content detected after fermentation was completed, the fermentation broth was dried at 40° C., and sampled and crushed according to Example 4 of the present invention; and

FIG. 7 is a data comparison chart of determination results of the triterpenoids content according to Example 4 of the present invention.

### DETAILED DESCRIPTION

The present invention provides an Antrodia cinnamomea strain with high triterpenoids yield and a use thereof. In order to make the objectives, technical solutions and effects of the present invention clearer and definite, the present invention is further described in detail below. It should be understood that, the specific embodiments described herein are merely used to illustrate the present invention, but not intended to limit the present invention.

Specifically, the method for breeding a novel Antrodia cinnamomea strain with a high triterpenoids yield through ultraviolet-induced mutation of a protoplast includes the following steps:

1) Preparation of Original Strain Mycelium

An Antrodia cinnamomea strain ATCC 200183 preserved in the laboratory is inoculated on an MEA slant, cultured in a thermostatic incubator at 26° C. in dark for 8 to 10 days, and aseptically sampled by a puncher, and the sample is placed in a shake-flask, and cultured for 3 to 5 days at a constant temperature of 26° C. at 120 rpm to obtain the original strain mycelium.

2) Preparation of Original Strain Protoplast

The shake-flask mycelium in Step 1) is centrifuged and the supernatant is discarded. An osmotic stabilizer of 10 volumes is added, the mixture is centrifuged after fully vibration, and the process is repeated twice. After the supernatant is discarded, a composite enzyme solution is added to the mycelium precipitate at a ratio of enzymatic hydrolysate:mycelium of 3:1 (mN), and the mixture is fully vibrated and cultured for 3 hr at 30° C. for enzy molysis. After enzy molysis is completed, the enzymatic hydrolysate is immediately distributed into 50 ml sterile centrifuge tubes, and centrifuged for 10 min at 4° C. and 3000 rpm. The precipitate is washed three times with an osmotic stabilizer of 10-time volume, so as to completely remove the enzymatic hydrolysate. After the stabilizer is resuspended, the mixture is centrifuged for 10 min at 1800 rpm, and the precipitate is discarded. The supernatant is further centrifuged for 10 min at 600 rpm, the supernatant is transferred, and the precipitate is discarded. Finally, the supernatant is centrifuged for 10 min at 3500 rpm, so as to obtain a high-purity Antrodia cinnamomea protoplast.

3) Protoplast Mutation

The precipitate obtained in Step 2) is dissolved with an osmotic stabilizer, counted by using a blood count board, the cell concentration is adjusted to 1×10⁵ to 5×10⁵ per ml, and the solution is formulated on the day to use. 5 sterile Petri dishes having a diameter of 9 cm are taken, 3 to 5 ml protoplast suspension is poured into each dish, and under electromagnetic stirring, the dishes are radiated by an ultraviolet lamp for 30″, 60″, 90″, 120″ and 150″ respectively, where the power of the ultraviolet lamp is 15 to 30 W, and the radiation distance is 25 to 35 cm; after radiation, the protoplast is immediately coated on a regeneration flat-plate for regeneration, and is subjected to primary screening according to the size of the regenerated colony. 10 to 30 single colonies of mycelium that grows fast are selected for shake-flask rescreening, the total triterpenoids content of the fermented mycelia is deter mined by using an oleanolic acid method, where 5 to 10 strains RT00X with high triterpenoids output are selected.

4) Verification of Stability of Genetic Properties

The 5 to 10 high-yield strains obtained through mutation screening are continuously passaged for 20 to 30 generations, and the mycelium growth status on an MEA flat-plate and the total triterpenoids output of shake-flask fermented mycelia are exa mined to finally screen out a new strain having stable properties, a high triterpenoids yield and growing rapidly.

The MEA solid medium in Step 1) and Step 3) is: 1% to 3% of glucose, 15% to 3% of malt extract, 0.1% to 0.3% of peptone, 1.5% to 2.0% of agar, with the rest being water, at a natural pH value, and moist heat sterilization for 20 min.

The liquid medium in Step 1) is: 1% to 3% of glucose, 1% to 3% of malt extract, 0.1% to 0.3% of peptone, with the rest being water, at a natural pH value, and moist heat sterilization 20 min.

The osmotic stabilizer in Steps 2) and 3) are 0.6 mol/L mannitol after sterilization by using a disposable syringe filter.

The composite enzyme solution in Step 2) contains, in mass concentration, 2.0% of lyticase, 0.5% of snailase and 0.5% of cellulase, the solid enzymes are weighed in proportion and dissolved in 0.6 mol/L mannitol, filtered and sterilized, and stored at −20° C.

The primary screening conditions in Step 3) are: on an MEA medium, culturing for 8 to 10 days at 26° C., and selecting a single colony having a large diameter as inoculums for rescreening.

The shake-flask rescreening conditions in Step 3) are:

The medium composition includes: 3.0% of bran, 2.5% of corn flour, 0.05% of magnesium sulfate, 0.3% (m/v) of cod liver oil, with the rest being water, and at a pH value of 2.9 to 3.1.

The medium formulation method is: boiling bran for 30 min over a low heat, and the filtrate is taken; after being gelatinized for 10 min at 100° C., corn flour is mixed with the bran filtrate; to the mixture, magnesium sulfate and cod liver oil are added, and concentrated hydrochloric acid is added to adjust the pH value of the solution to 2.9 to 3.1; the medium is distributed at a load of 80 ml/200 ml, and then subjected to moist heat sterilization for 20 min at 121° C.

The culture conditions are: at 26° C., at arotation speed of 90 rpm, the inoculum is logarithmic phase liquid inoculum, the inoculum concentration is 5%, and the culturing time is 4 days.

The MEA solid medium used in Step 4) is the same as that in Step 1), and the liquid medium used is the same as the rescreening medium in Step 3).

In Step 3), oleanolic acid method is adopted to deter mine the total triterpenoids content of the fermented mycelia. The principle is: oleanolic acid is a five-membered cyclic triterpenoids, and undergoes chromogenic reactions with various color-developing agents for colorimetric assay. Oleanolic acid has chromogenic reactions with vanillic aldehyde-perchloric acid reagent and vanillic aldehyde-sulfuric acid reagent and exhibits purple color. There is a linear relationship between the concentration and the absorbance of oleanolic acid, which accords with the Lambert-Beer's Law and can be used in colorimetric assay. The adopted instruments and reagents are as follows:

1. Instruments: 752-ultraviolet spectrophotometer, electric-heated thermostatic water bath, analytical balance, electric-heated thermostatic dry box, and stoppered tubes.

2. Reagents: vanillic aldehyde, glacial acetic acid, absolute ethyl alcohol, ethyl acetate and perchloric acid (all are analytical grade), and oleanolic acid standard.

Determination of triterpenoids contents by adopting the oleanolic acid method includes the following operating steps:

1. Preparation of oleanolic acid standard: 10mg oleanolic acid standard is precisely weighed, and dissolved with absolute ethyl alcohol as solvent, to formulate 100 ml ethanol solution of oleanolic acid having a concentration of 0.1 mg/ml.

2. Preparation of vanillic aldehyde-glacial acetic acid solution: 0.552g vanillic aldehyde is precisely and rapidly weighed, dissolved with a suitable amount of glacial acetic acid, immediately poured into a 10-ml volumetric flask, diluted with glacial acetic acid to the scale for use on the same day.

3. Drafting a standard curve: 0.1, 0.2, 0.3, 0.4 and 0.5ml oleanolic acid standard solutions are precisely extracted and respectively placed in stoppered tubes, heated to remove the solvent through evaporation, 0.4 ml newly formulated 5% vanillic aldehyde-glacial acetic acid solution and 1.5 ml perchloric acid are added. The mixture is heated for 15 min in a thermostatic water bath of 70° C., and cooled to room temperature with flowing water, and then 5 ml ethyl acetate is added for dilution, the mixture is fully shaken, and then the absorbance value is deter mined at a wavelength of 560 nm.

4. Sample extraction: 1 g sample powder is added to 30 ml absolute ethyl alcohol, the mixture is refluxed for 1.5 h in a water bath of a thermostatic water bath of 90° C., repeated for three times, and the filtrates are combined and diluted to 100 ml.

5. Sample determination: 0.1 ml sample is precisely measured, heated to remove the solvent through evaporation, 0.4 ml newly formulated 5% vanillic aldehyde-glacial acetic acid solution and 1.5 ml perchloric acid are added, heated for 15 min in a thermostatic water bath of 70° C., and cooled to room temperature with flowing water, and then 5 ml ethyl acetate is added for dilution, the mixture is fully shaken, and then the absorbance value is deter mined at a wavelength of 560 nm.

6. In the blank test, maximum 0.5 ml absolute ethyl alcohol is used as the reference solution, and the oleanolic acid content in the sample is calculated according to the standard curve drafted in Step 3 and is converted into percentage content.

The novel Antrodia cinnamomea strain with a high triterpenoids yield obtained through the mutation breeding method is classified as Antrodia cinnamomea, and deposited with China Center for Type Culture Collection (CCTCC) with a deposition date of Aug. 2, 2013 and an accession number of CCTCC M2013359.

In the following, the present invention is further described through examples.

### Example 1

According to the mutation breeding method of the present invention, a novel Antrodia cinnamomea strain with a high triterpenoids yield is obtained through mutation breeding.

The mutation process includes the following steps:

1.1 Preparation of Original Strain Mycelium

An original Antrodia cinnamomea strain ATCC 200183 was inoculated on an MEA slant, cultured for 8 days in a thermostatic incubator of 26° C. in dark, and aseptically sampled by a puncher, and the sample was placed in a shake-flask, and cultured for 5 days at a constant temperature of 26° C. at 120 rpm, to obtain 300 ml fermentation broth of original strain mycelium.

The MEA slant medium was: 2% of glucose, 2% of malt extract, 0.1% of peptone, 1.5% of agar, with the rest being water, at a natural pH value, and moist heat sterilization for 20 min; liquid medium was: 2% glucose, 2% malt extract, 0.1% peptone, with the rest being water, at a natural pH value, and moist heat sterilization for 20 min.

1.2 Preparation of Original Strain Protoplast

The fermentation broth obtained in 1.1 was centrifuged. The supernatant was discarded to obtain about 25 g mycelium pellet precipitate. 250 ml mannitol having a concentration of 0.6 mol/L was added. The mixture was centrifuged for 10 min at 5000 rpm after fully vibration, and the process was repeated twice. After the supernatant was discarded, about 80 ml composite enzyme solution was added to the mycelium precipitate, fully vibrated and cultured for 3 h at 30° C. for enzy molysis. After enzy molysis was completed, the enzymatic hydrolysate was immediately distributed into 50 ml sterile centrifuge tubes, and centrifuged for 10 min at 4° C. and 3000 rpm. The precipitate was washed with sufficient amount of 0.6 mol/L mannitol three times so as to completely remove the enzyme solution. After mannitol was resuspended, the mixture was centrifuged for 10 min at 1800 rpm, and the precipitate was discarded. The supernatant was further centrifuged for 10 min at 600 rpm, the supernatant was transferred, and the precipitate was discarded. Finally, the supernatant was centrifuged for 10 min at 3500 rpm to obtain the high-purity Antrodia cinnamomea protoplast.

The composite enzyme solution containing, in mass concentration, 2.0% of lyticase, 0.5% of snailase and 0.5% of cellulase, and the solid enzyme was weighed at proportion and dissolved in 0.6 mol/L mannitol, and stored at −20° C. after filtration and sterilization.

1.3 Protoplast Mutation

The precipitate obtained in 1.2 was dissolved with an osmotic stabilizer, counted by using a blood count board, the cell concentration was adjusted to 5.0×10⁵ per ml, and the solution is formulated on the day to use. 5 sterile Petri dishes having a diameter of 9 cm were taken, and 5 ml protoplast suspension was poured into each dish. Under electromagnetic stirring, the dishes were radiated by an ultraviolet lamp for 30″, 60″, 90″, 120″ and 150″, where the power of the ultraviolet lamp was 18 W, and the radiation distance is 32 cm; after radiation, the protoplast was immediately coated on a regeneration flat-plate for regeneration and cultured for 8 days at 26° C., and was subjected to primary screening according to the size of the regenerated colony. 28 single colonies of mycelium that grows fast were selected for shake-flask rescreening, which were respectively labeled as RT001, RT002, RT003 . . . RT0027 and RT0028, the total triterpenoids content of the fermented mycelia was deter mined by using an oleanolic acid method, and 6 strains with high triterpenoids outputs were selected, which were labeled as RT002, RT009, RT0017, RT0023, RT0024 and RT0027 respectively.

The shake-flask rescreening conditions included the rescreening medium composition, the medium formulation method and the culture conditions, which were specifically as follows:

The medium composition was: 3.0% of bran, 2.5% of corn flour, 0.05% of magnesium sulfate, 0.5% (m/v) of cod liver oil, with the rest being water, and at a pH value of 2.95.

The medium formulation method was: boiling bran for 30 min over a low heat, and the filtrate was taken; after being gelatinized for 10 min at 100° C., corn flour was mixed with the bran filtrate; to the mixture, magnesium sulfate and cod liver oil were added, and concentrated hydrochloric acid was added to adjust the pH value of the solution to 2.95; the medium was distributed at a load of 50 ml/200 ml, and then subjected to moist heat sterilization for 20 min at 121° C.

The fermentation conditions were: at 26° C., at arotation speed of 90 rpm, the inoculum was logarithmic phase liquid inoculum, the inoculum concentration is 5%, and the fermentation time was 4 days.

1.4 Verification of Stability of Genetic Properties

The 6 high-yield strains obtained through mutation screening were continuously passaged for 30 generations, and according to the mycelium growth status on an MEA flat-plate and the total triterpenoids output of shake-flask fermented mycelia, a new strain RT0023 having stable properties, having a high triterpenoids yield and growing rapidly was finally screened out. This strain RT0023 is the strain with accession number of CCTCC M2013359.

The MEA flat-plate medium and the shake-flask fermentation conditions in the passage were the same as the rescreening conditions.

The properties of the strain RT0023 are described below:

The Antrodia cinnamomea strain were cultured in dark, has clap connection under observation by microscope, can produce spores, and can generate a large amount of triterpenoids under suitable liquid culturing condition and solid culturing condition. The strain grows slower on a PDA flat-plate and grows faster on an MEA solid flat-plate. Suitable growth temperature is 22 to 28° C., and the optimum growth temperature is 26° C., and strain grows very slowly at a temperature lower than 10° C., and hardly grows at a temperature higher than 32° C. On an MEA flat-plate, the strain evenly covers the whole flat-plate with an inoculation dot as the center, and the time need for the strain to grow all over the flat-plate on a Petri dish having a diameter of 9 cm at 26° C. is about 20 to 22 days, the color of the mycelium is white at the beginning, and then slowly turns to be pale yellow to orange. During the growth of the mycelium, since volatilesubstances are generated, special fruity flavor and camphor flavor are produced. Under the shake-flask liquid vibration culture conditions, the strain can produce mycelium pellets of different size, the fermentation broth has special peach flavor; under static culture conditions, orange mycelium grows all over the liquid surface.

### Example 2

An original strain A (ATCC 200183) and a mutant strain B (CCTCC M2013359) were respectively used as the fermentation strains, and fermented under shake-flask Antrodia cinnamomea triterpenoids conditions. The specific implementation is as follows:

2.1 Slant Strain Activation

Slant strains of the Antrodia cinnamomea original strain A and the mutant strain B were respectively inoculated on an MEA flat-plate medium for activation culture, where the culture temperature was 26° C., and the culture time was 15 days.

2.2 Preparation of Shake-Flask Liquid Inoculum

Under sterile conditions, 5 mycelium blocks of the original strain A and the mutant strain B were sampled by a puncher respectively, and transferred into a liquid shake-flask for liquid inoculum culture, where the culture temperature was 26° C., the rotation speed is 100 rpm, and the culture time was 6 days.

The liquid inoculums medium composition was: 1.0% of glucose, 1.0% of malt extract, 0.1% of peptone, 0.05% of magnesium sulfate, with the rest being water, at a natural pH value, 121° C., and moist heat sterilization for 20 min.

2.3 Fermentation Culture

The shake-flask inoculums of the two strains obtained in 2.2 were respectively transferred into a conical flask at an inoculum concentration of 5% for fermentation culture, where the conical flask had a load of 500 ml. Each strain had 5 parallel samples, which were respectively labeled as A1, A2, A3, A4, A5, B1, B2, B3, B4 and B5, where Ax corresponds to the original strain, and Bx corresponds to the mutant strain.

The fermentation medium composition was: 3.0% of bran, 2.5% of corn flour, 0.05% of magnesium sulfate, 0.5% (m/v) of cod liver oil, with the rest being water, at a pH value of 2.95.

The medium formulation method was: boiling bran for 30 min over a low heat, and the filtrate was taken; after being gelatinized for 10 min at 100° C., corn flour was mixed with the bran filtrate; to the mixture, magnesium sulfate and cod liver oil were added, and concentrated hydrochloric acid was added to adjust the pH value of the solution to 3.1, the medium was distributed at a load of 500 ml/2000 ml, and then subjected to moist heat sterilization for 20 min at 121° C.

The culture conditions were: the culture temperature is 26° C., the initial pH value is 3.1, the rotation speed is 90 rpm, and culture is continuously carried out for 4 days under these conditions.

2.4 Determination of Mycelium and Triterpenoids Output

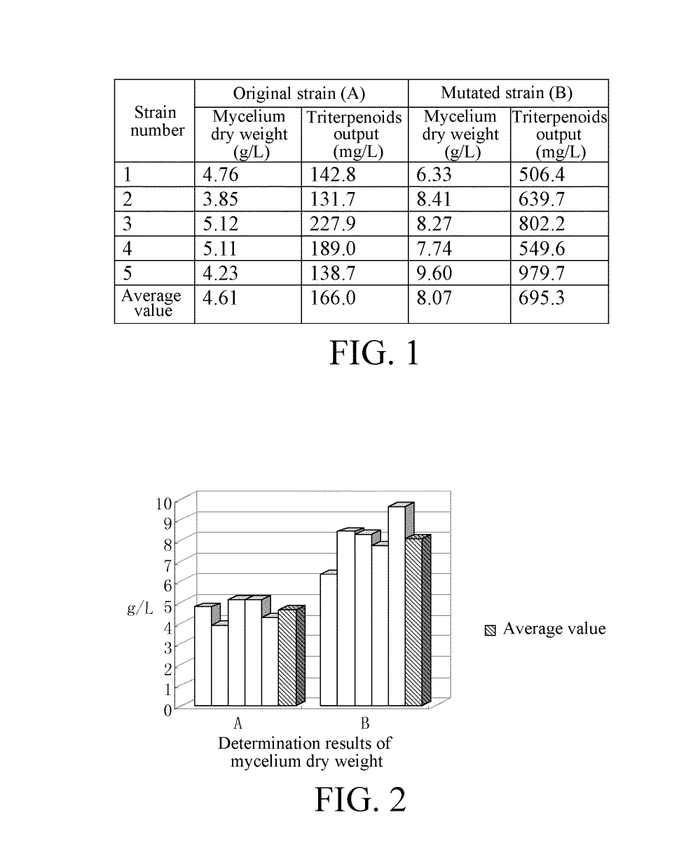

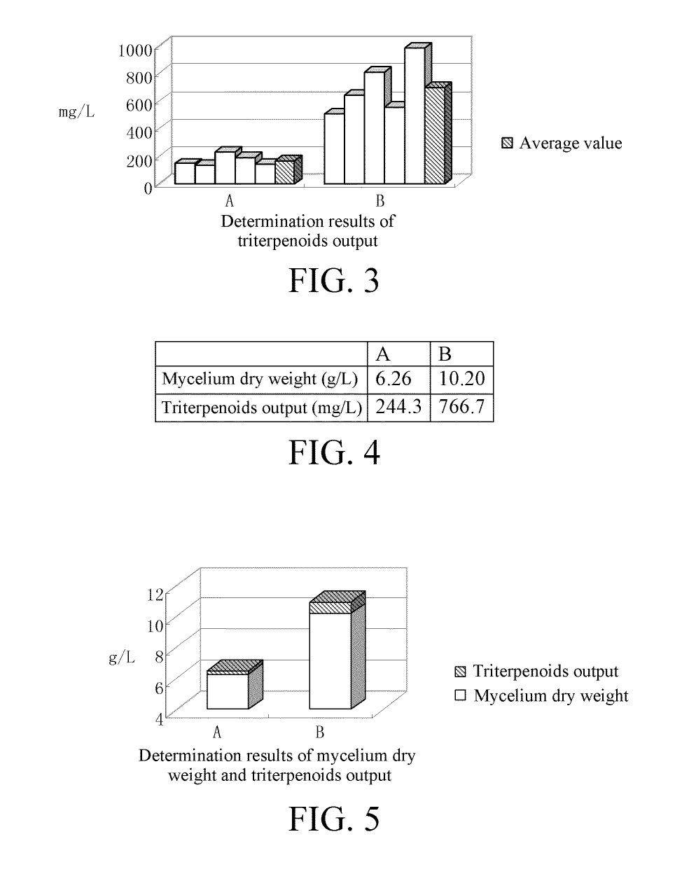

After fermentation was completed, mycelia were respectively collected through centrifugation according to the numbers, and dried at 40° C., the mycelium dry weight and the triterpenoids content of each fermentation broth were respectively deter mined, and the obtained mycelium dry weight and triterpenoids content were converted into the mycelium dry weight and triterpenoids output per liter of fermentation broth. The specific results are shown in FIG. 1, where the chart of the mycelium dry weight is shown in FIG. 2, and the chart of the triterpenoids output is shown in FIG. 3.

### Example 3

An original strain A (ATCC 200183) and a mutant strain B (CCTCC M2013359) were respectively used as the fermentation strains, and fermented in a 500L fermentation tank. The specific implementation is as follows:

3.1 Slant Strain Activation

Slant strains of the original strain A and the mutant strain B were respectively inoculated on an MEA flat-plate medium for activation culture, where the culture temperature was 26° C., and the culture time was 15 days.

3.2 Preparation of Shake-Flask Liquid Inoculum

Under sterile conditions, 5 mycelium blocks of the original strain A and the mutant strain B were sampled by a puncher respectively, and transferred into a liquid shake-flask for liquid inoculum culture, where the culture temperature was 26° C., the rotation speed is 100 rpm, and the culture time was 6 days.

The liquid inoculums medium composition was: 1.0% of glucose, 1.0% of malt extract, 0.1% of peptone, 0.05% of magnesium sulfate, with the rest being water, at a natural pH value and 121° C., and moist heat sterilization for 20 min.

3.3 Inoculum Propagation

The shake-flask inoculums A and B obtained in 3.2 were respectively inoculated into a 50 L fermentation tank at an inoculum concentration of 10% for inoculum propagation. The inoculum tank medium composition was: 5.0% of bran, 4.5% of corn flour, 0.05% of magnesium sulfate, and with the rest being water. The culture conditions were: the culture temperature was 26° C., the initial pH value was a natural pH value, the rotation speed was 110 rpm, the ventilation ratio was 1.0 vvm, and culture was continuously carried out for 5 days under these conditions.

The medium formulation method was: boiling bran for 30 min over a low heat, and the filtrate was taken; after being gelatinized for 10 min at 100° C., corn flour was mixed with the bran filtrate; to the mixture, magnesium sulfate was added, the pH value was a natural value, the medium was distributed at a load of 35 L/50 L, and then subjected to moist heat sterilization for 20 min at 121° C.

3.4 Fermentation Culture

The inoculum tank inoculums obtained in 3.3 were transferred into a 500 L fermentation tank at an inoculum concentration of 10% for triterpenoids process culture. The fermentation tank medium composition was: 2.2% of bran, 2.0% of corn flour, 0.05% of magnesium sulfate, 0.5% (m/v) of cod liver oil, with the rest being water, at a pH value of 3.0. The culture conditions were: the culture temperature was 26° C., the initial pH value was 3.0, the rotation speed was 90 rpm, the ventilation ratio was 0.6 vvm, and culture was continuously carried out for 65 hours under these conditions.

The medium formulation method was: boiling bran for 30 min over a low heat, and the filtrate was taken; after being gelatinized for 10 min at 100° C., corn flour was mixed with the bran filtrate; to the mixture, magnesium sulfate and cod liver oil were added, and concentrated hydrochloric acid was added to adjust the pH value of the solution to 3.0, the medium was distributed at a load of 350 L/500 L, and then subjected to moist heat sterilization for 20 min at 121° C.

3.5 Determination of Mycelium and Triterpenoids Output

After fermentation was completed, mycelia were collected through frame filtration, and dried at 40° C., the mycelium dry weight and the triterpenoids content of each fermentation broth were respectively deter mined, and the obtained mycelium dry weight and triterpenoids content were converted into the mycelium dry weight and triterpenoids output per liter of fermentation broth. The specific results are shown in FIG. 4 and FIG. 5.

### Example 4

An original strain A (ATCC 200183) and a mutant strain B (CCTCC M2013359) were respectively used as the fermentation strains, and fermented in a 500 L fermentation tank. The specific implementation is as follows:

4.1 Slant Strain Activation

Slant strains of the Antrodia cinnamomea original strain A and the mutant strain B were respectively inoculated on an MEA flat-plate medium for activation culture, where the culture temperature was 26° C., and the culture time was 15 days.

4.2 Preparation of Shake-Flask Liquid Inoculum

Under sterile conditions, 5 mycelium blocks of the original strain A and the mutant strain B were sampled by a puncher respectively, and transferred into a liquid shake-flask for liquid inoculum culture, where the culture temperature was 26° C., the rotation speed is 100 rpm, and the culture time was 6 days.

The liquid inoculums medium composition was: 1.0% of glucose, 1.0% of malt extract, 0.1% of peptone, 0.05% of magnesium sulfate, with the rest being water, at a natural pH value and 121° C., and moist heat sterilization 20 min.

4.3 Solid Fermentation

The shake-flask inoculums obtained in 4.2 were respectively inoculated into a solid medium, and fully shaken for solid fermentation. Each strain had 5 parallel samples, which were respectively labeled as A1, A2, A3, A4, A5, B1, B2, B3, B4 and B5. The solid medium composition was: 85% of bran, 14.5% of corn flour, 0.1% of magnesium sulfate, 0.4% of olive oil, diluted hydrochloric acid at a pH value of 3.0 was used for mixing, the ratio of material to solvent was 1.05:1. The formulated medium was charged into a 2 L conical flask at a load of 450 g. The fermentation conditions were: the inoculum concentration was 5% (5 ml liquid inoculum is inoculated per 100 g wet material), and culture was carried out for 10 days at 25° C.

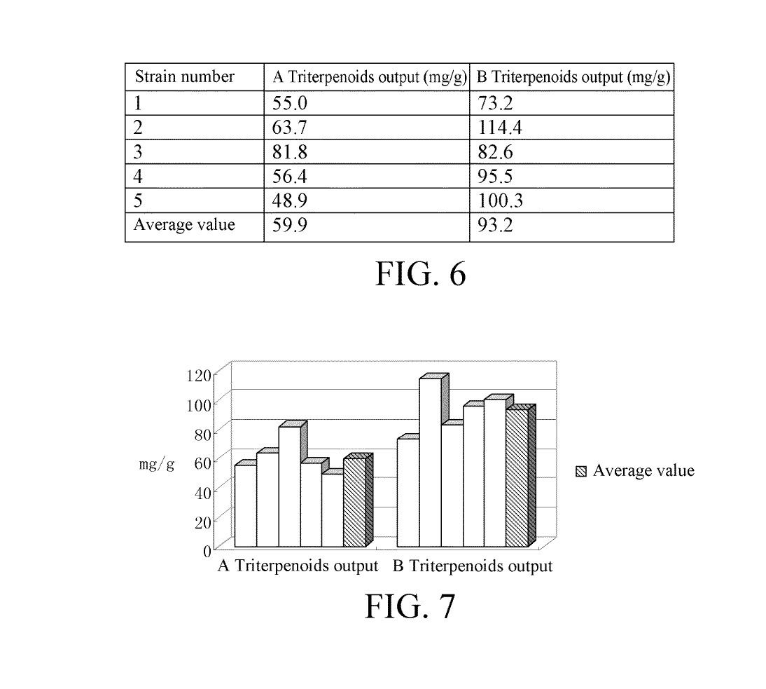

After fermentation was completed, the culture in each conical flask was dried at a low temperature of 40° C., and then sampled and crushed, and the triterpenoids content was detected. The specific results are shown in FIG. 6 and FIG. 7.

In view of the above, it can be seen from Examples 2 to 4 that, as for the mycelium of this strain (CCTCC M2013359), the growth rate is fast, the triterpenoids output is high, and the genetic properties are stable, and the strain mycelium is suitable for industrialized fermentation. Compared with the original strain ATCC 200183, the mycelium output under liquid culturing condition is increased by more than 1.5 times; the intracellular triterpenoids output is increased by 2 to 3 times; the fermentation period is shortened to 65 hours compared with the original of more than 10 days; the total triterpenoids output under a solid culturing condition is increased by 1 to 2 times; and the fermentation period is shortened to 10 days compared with the original of more than 90 days.

It should be understood that the use of the present invention is not limited to the examples above. Persons of ordinary skill in the art can make improvements or alternations according to the description above, and all these improvements and alternations shall fall within the protection scope of the appended claims of the present invention.

What is claimed is:

1. A triterpenoids high yielding strain of Antrodia cinnamomea deposited under the accession number of CCTCC M2013359.

2. Use of the triterpenoids high yielding strain of Antrodia cinnamomea according to claim 1 for producing triterpenoids through fermentation.
